(19) United States
(12) Patent Application Publication
(10) Pub. No.: US 2012/0277568 A1
(43) Pub. Date: Nov. 1, 2012

(54) WIRELESS INTRAOCULAR PRESSURE MONITORING DEVICE, AND SENSOR UNIT AND READER UNIT THEREOF

(75) Inventors: Jin-Chern Chiou, Hsinchu City (TW); Chien-Kai Tseng, Miaoli County (TW)

(73) Assignee: National Chiao Tung University, Hsinchu (TW)

(21) Appl. No.: 13/342,414

(22) Filed: Jan. 3, 2012

(30) Foreign Application Priority Data
Apr. 28, 2011 (TW) 100114832

(51) Int. Cl. A61B 3/16

(52) U.S. Cl. 600/398

(57) ABSTRACT

A wireless intraocular pressure monitoring device includes a sensor unit and a reader unit. The sensor unit includes: a soft contact lens for wearing on a cornea such that a curvature of the soft contact lens corresponds substantially to that of the cornea; an inductor embedded in the soft contact lens and having an inductance that corresponds to intraocular pressure when the soft contact lens is worn on the cornea; and a wireless transceiver module operable to generate an oscillation signal having a frequency dependent on the inductance of the inductor and to wirelessly transmit the oscillation signal. The reader unit is operable to receive and convert the oscillation signal into an output signal corresponding to the intraocular pressure.

### CROSS-REFERENCE TO RELATED APPLICATION

This application claims priority of Taiwanese Application No. 100114832, filed on Apr. 28, 2011.

### BACKGROUND OF THE INVENTION

1. Field of the Invention

The present invention relates to an intraocular pressure monitoring device, more particularly to a wireless intraocular pressure monitoring device.

2. Description of the Related Art

Glaucoma, one of the eye diseases that lead to vision loss, may be categorized into chronic simple glaucoma and acute congestive glaucoma. Chronic simple glaucoma, also known as open-angle glaucoma, accounts for approximately 90% of the cases in the U.S., is characterized by a gradual rise in intraocular pressure, and does not cause pain. On the other hand, acute congestive glaucoma, also known as narrow-angle glaucoma, is less common and is characterized by a sudden rise in intraocular pressure generally attributed to blockage of the drainage route of the aqueous humor. Symptoms associated with this type of glaucoma may be alleviated by using certain types of eye drops to improve drainage of the aqueous humor and/or using certain drugs (e.g., diuretics) to suppress secretion of the aqueous humor.

According to the statistics of the World Health Organization (WHO), there are approximately 67 million cases of glaucoma worldwide, among which 6.4 million cases progressed to complete vision loss. Moreover, the elderly accounts for 75% of the 6.4 million cases. The statistics further show that, among the population of age 40 or above (currently 3 million people), 0.12 million people suffered vision loss due to glaucoma.

Currently, glaucoma cannot be cured, and symptoms and development of which can only be suppressed through the use of drugs and/or through surgical operations, which aim to reduce the intraocular pressure so as to prevent damage to the optic nerves. Relevant researches further indicate that the main cause of glaucoma has been the variation in intraocular pressure, and that people who are diagnosed with diabetes, who have high blood pressure or myopia, and who have family members suffering from glaucoma are at high risk of developing glaucoma.

Therefore, timely control of intraocular pressure is the most important part of controlling the development of glaucoma. That is to say, regular monitoring of intraocular pressure is important, especially in finding out the cause of a rise in the intraocular pressure of a patient. However, since access to relevant medical equipments is generally limited due to their prices and sizes, people with glaucoma are generally unaware of their own biological statuses.

Referring to FIG. 1, U.S. Pat. No. 7,137,952 discloses a conventional non-invasive wireless intraocular pressure monitoring device including a sensor unit 1, an interrogation unit 14, a wireless receiver unit 15, and a computer device 16.

The sensor unit 1 includes a soft contact lens 101 made of silicone, an active-type resistive strain gauge 10, a passive-type resistive strain gauge 11, a low-power transponder 12, and an antenna 13. The active-type and passive-type resistive strain gauges 10, 11 are arranged to form a Wheatstone bridge structure and are embedded in the soft contact lens 101. The active-type resistive strain gauge 10 has a resistance that varies based on a variation in curvature of the soft contact lens 101, which may be caused by a change in curvature of the cornea attributed to a change in the intraocular pressure. The passive-type resistive strain gauge 11, on the other hand, is operable to provide a temperature-based compensation for correcting errors associated with the variation in the resistance. Next, the resistance may be converted into a sensor voltage corresponding to the intraocular pressure.

The lower-power transponder 12 is connected electrically to the Wheatstone bridge structure, and is operable to perform a first modulation process upon the sensor voltage so as to generate a first carrier-frequency signal for wireless transmission to the interrogation unit 14 via the antenna 13.

The interrogation unit 14 is operable to wirelessly power the low-power transponder 12, to wirelessly receive the first carrier-frequency signal from the low-power transponder 12, and to perform a first demodulation process upon the first carrier-frequency signal received thereby so as to obtain a demodulated voltage corresponding to the sensor voltage. The interrogation unit 14 is further operable to perform an analog-to-digital conversion process upon the demodulated voltage so as to obtain a digital signal corresponding to the sensor voltage, and to subsequently perform a second modulation process upon the digital signal so as to obtain a second carrier-frequency signal for wireless transmission to the wireless receiver unit 15 via an antenna.

The wireless receiver unit 15 is operable to wirelessly receive the second carrier-frequency signal from the interrogation unit 14, and to perform a second demodulation process upon the second carrier-frequency signal received thereby so as to obtain demodulated data corresponding to the sensor voltage.

The computer device 16 is connected electrically to the wireless receiver unit 15 for receiving the demodulated data therefrom, and is operable to output an intraocular pressure value based on the demodulated data with reference to a conversion table that defines a plurality of relationships between a plurality of data values and a plurality of corresponding intraocular pressure values, respectively.

However, the conventional non-invasive wireless intraocular pressure monitoring device has the disadvantages of: poor sensitivity (in the order of μV's) attributed to the limited range of variation of the resistance of the active-type resistive strain gauge 10, such that the range of variation of the sensor voltage is, as a result, rather limited; poor measurement accuracy (i.e., low SNR) attributed to the rather limited range of variation of the sensor voltage, and to losses that arise from the analog-to-digital conversion process, the first and second modulation processes, and the first and second demodulation processes; uncomfortable for long-duration wearing due to the hydrophobic property of silicone, and hence not suitable for long-duration continuous monitoring of intraocular pressure; and high costs of hardware attributed to complex modulating components of the low-power transponder 12, the interrogation unit 14, and the wireless receiver unit 15.

### SUMMARY OF THE INVENTION

Therefore, an object of the present invention is to provide an intraocular pressure monitoring device capable of alleviating the aforesaid drawbacks of the prior art.

Accordingly, a wireless intraocular pressure monitoring device of the present invention includes a sensor unit and a reader unit.

The sensor unit includes: a soft contact lens for wearing on a cornea such that a curvature of the soft contact lens corresponds substantially to that of the cornea; an inductor embedded in the soft contact lens and having an inductance that corresponds to intraocular pressure when the soft contact lens is worn on the cornea; and a wireless transceiver module coupled electrically to the inductor, and operable to generate an oscillation signal having a frequency dependent on the inductance of the inductor and to wirelessly transmit the oscillation signal.

The reader unit is operable to wirelessly receive the oscillation signal, and to convert the oscillation signal into an output signal corresponding to the intraocular pressure.

### BRIEF DESCRIPTION OF THE DRAWINGS

Other features and advantages of the present invention will become apparent in the following detailed description of the preferred embodiment with reference to the accompanying drawings, of which:

FIG. 1 is a block diagram to illustrate a conventional wireless intraocular pressure monitoring device;

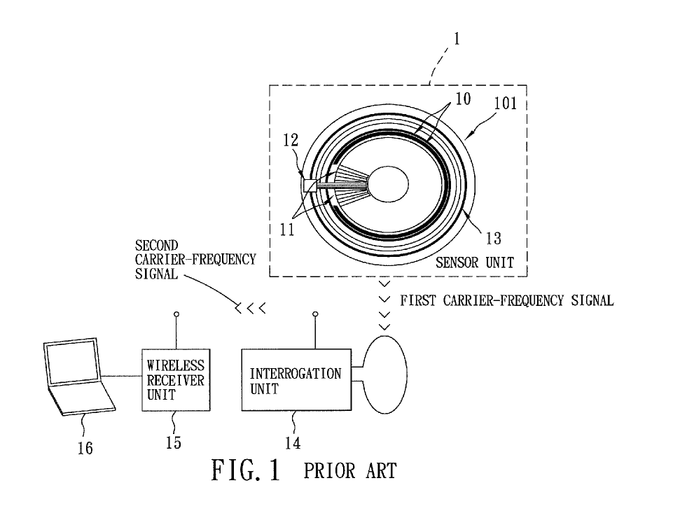

FIG. 2 is a block diagram to illustrate the preferred embodiment of a wireless intraocular pressure monitoring device according to the present invention;

FIG. 3 is a block diagram to illustrate components of a sensor unit of the wireless intraocular pressure monitoring device of the preferred embodiment; and

FIG. 4 is a block diagram to illustrate components of a frequency-to-voltage converter of a reader unit of the wireless intraocular pressure monitoring device of the preferred embodiment.

### DETAILED DESCRIPTION OF THE PREFERRED EMBODIMENT

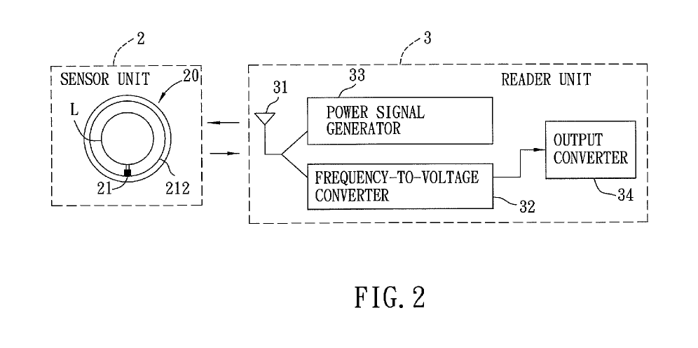

Referring to FIG. 2, the preferred embodiment of a wireless intraocular pressure monitoring device according to the present invention includes a sensor unit 2 and a reader unit 3.

The sensor unit 2 includes a contact lens 20 (e.g., a soft contact lens), an inductor “L”, and a wireless transceiver module 21.

The contact lens 20 is for wearing on a cornea such that a curvature of the contact lens 20 corresponds substantially to that of the cornea, and is preferably made of Hydroxyethylmethacrylate (HEMA) for high oxygen permeability and comfortable long-duration wearing.

The inductor “L” may be a ring-shaped inductor embedded in the contact lens 20 and having an inductance that corresponds to a radius of the inductor “L”. Since the contact lens 20 conforms in surface shape to the cornea, a variation in the curvature of the cornea attributed to a variation in the intraocular pressure will cause a corresponding variation in the curvature of the contact lens 20, which, in turn, causes a corresponding variation in the radius of the inductor “L”. Thus, the inductance of the inductor “L” corresponds to the intraocular pressure when the contact lens 20 is worn on the cornea.

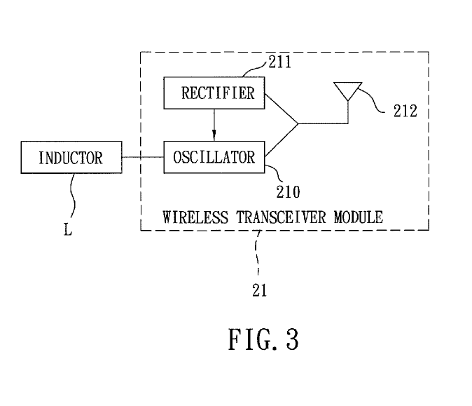

Referring to FIG. 3, the wireless transceiver module 21 is preferably embedded in the contact lens 20, is coupled electrically to the inductor “L”, and is wirelessly powered through electromagnetic inductance to generate an oscillation signal having a frequency dependent on the inductance of the inductor “L” and to wirelessly transmit the oscillation signal.

The wireless transceiver module 21 includes an oscillator 210, a rectifier 211, and a first wireless transceiver element 212. In this embodiment, the wireless transceiver module 21 is realized by a radio frequency integrated circuit (RFIC). However, implementation and configuration of the wireless transceiver module 21 is not limited to such.

The oscillator 210 is coupled electrically to the inductor “L”, and includes a capacitor (not shown) that has a predetermined, fixed capacitance and that cooperates with the inductor “L” to form an LC oscillator for generating the oscillation signal, and is further coupled electrically to the first wireless transceiver element 212 for wirelessly transmitting the oscillation signal through the first wireless transceiver element 212. It is worth noting that the oscillation signal thus generated has a frequency suitable for wireless radio frequency transmission, such that a modulation process is not needed.

The rectifier 211 is coupled electrically to the first wireless transceiver element 212 for wirelessly receiving a power signal through the first wireless transceiver element 212, and is further coupled electrically to the oscillator 210 for providing power to the oscillator 210.

The first wireless transceiver element 212 is an antenna in this embodiment, and may be otherwise in other embodiments.

The reader unit 3 is operable to generate and wirelessly transmit the power signal, to wirelessly receive the oscillation signal, and to convert the oscillation signal into an output signal corresponding to the intraocular pressure. In this embodiment, the reader unit 3 includes a second wireless transceiver element 31, a frequency-to-voltage converter 32, and a power signal generator 33. The second wireless transceiver element 31 is an antenna in this embodiment, and may be otherwise in other embodiments.

The power signal generator 33 is operable to generate the power signal, and is coupled electrically to the second wireless transceiver element 31 for wirelessly transmitting the power signal through the second wireless transceiver element 31 to power the sensor unit 2. The frequency-to-voltage converter 32 is coupled electrically to the second wireless transceiver element 31 for wirelessly receiving the oscillation signal through the second wireless transceiver element 31, and is operable to generate a converter output with a value corresponding to the frequency of the oscillation signal.

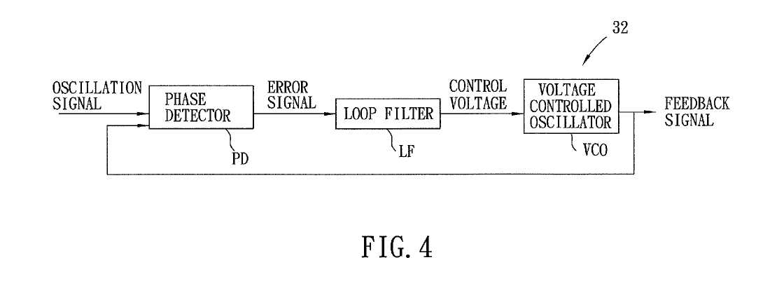

Referring to FIG. 4, in this embodiment, the frequency-to-voltage converter 32 includes a phase detector (PD), a loop filter (LF), and a voltage controlled oscillator (VCO).

The phase detector (PD) is operable to wirelessly receive the oscillation signal through the second wireless transceiver element 31, and to generate an error signal based on a difference in frequency and phase between the oscillation signal and a feedback signal.

The loop filter (LF) is connected electrically to the phase detector (PD) to receive the error signal from the loop filter (LF), and is operable to remove high frequency components and noise from the error signal through filtering so as to output a control voltage corresponding to the error signal. When the frequency and the phase of the oscillation signal match respectively those of the feedback signal, the frequency-to-voltage converter 32 is in a locked state in which the control voltage is non-varying, the frequency of the feedback signal is stabilized (i.e., no frequency drift), and the control voltage serves as the converter output. In this embodiment, the converter output is an analog voltage output serving as the output signal.

The voltage controlled oscillator (VCO) is connected electrically to the loop filter (LF) for receiving the control voltage therefrom, is operable to generate the feedback signal having a frequency and a phase that are dependent on a magnitude of the control voltage, and is further coupled electrically to the phase detector (PD) for providing the feedback signal to the phase detector (PD).

However, implementation of the frequency-to-voltage converter 32 is not limited to such. In other embodiments, the frequency-to-voltage converter 32 may be replaced by such as a frequency-to-current converter that outputs a current with a magnitude corresponding to the frequency of the oscillation signal and hence to the intraocular pressure.

In a modification where the converter output is an analog voltage output, and the value of the converter output is a magnitude of the analog voltage output, the reader unit 3 further includes an output converter 34 (see FIG. 2) coupled electrically to the frequency-to-voltage converter 32, and configured to perform analog-to-digital conversion upon the converter output to result in a digital signal corresponding to the frequency of the oscillation signal, and to obtain the output signal based on the digital signal via one of a predefined look-up table (i.e., a table of frequency vs. intraocular pressure) and a predefined mathematical algorithm (i.e., an equation defining a mathematical relation between frequency and intraocular pressure).

In summary, as the oscillator 210 generates the oscillation signal based on the inductance of the inductor “L”, any variation in the inductance of the inductor “L” is substantially amplified so that subtle variations in the intraocular pressure may be detected, thereby improving the signal-to-noise ratio. Further, since the number of modulation and demodulation processes involved in the generation of the converter output (or the digital signal) from the inductance of the inductor “L” is relatively reduced, measurements of the intraocular pressure thus obtained are relatively accurate and precise. Moreover, since the contact lens 20 is made of HEMA, which generally exhibits high oxygen permeability and increased hydrophilicity, it is suitable for long-duration wearing. Last, but not the least, since the oscillation signal is wirelessly and directly transmitted via the first wireless transceiver element 212 without being modulated onto a carrier signal, complex modulation components are not required, and hence the production cost is relatively low.

While the present invention has been described in connection with what is considered the most practical and preferred embodiment, it is understood that this invention is not limited to the disclosed embodiment but is intended to cover various arrangements included within the spirit and scope of the broadest interpretation so as to encompass all such modifications and equivalent arrangements.

What is claimed is:

1. A wireless intraocular pressure monitoring device comprising:
a sensor unit including
a soft contact lens for wearing on a cornea such that a curvature of said soft contact lens corresponds substantially to that of the cornea,
an inductor embedded in said soft contact lens and having an inductance that corresponds to intraocular pressure when said soft contact lens is worn on the cornea, and
a wireless transceiver module coupled electrically to said inductor, and operable to generate an oscillation signal having a frequency dependent on the inductance of said inductor and to wirelessly transmit the oscillation signal; and
a reader unit operable to wirelessly receive the oscillation signal, and to convert the oscillation signal into an output signal corresponding to the intraocular pressure.

2. The wireless intraocular pressure monitoring device as claimed in claim 1, wherein said wireless transceiver module includes:
a first wireless transceiver element; and
an oscillator coupled electrically to said inductor, including a capacitor that is operatively associated with said inductor for generating the oscillation signal, and further coupled electrically to said first wireless transceiver element for wirelessly transmitting the oscillation signal.

3. The wireless intraocular pressure monitoring device as claimed in claim 2, wherein said wireless transceiver module further includes
a rectifier coupled electrically to said first wireless transceiver element for receiving a power signal through said first wireless transceiver element, and further coupled electrically to said oscillator for providing power to said oscillator.

4. The wireless intraocular pressure monitoring device as claimed in claim 2, wherein said first wireless transceiver element is an antenna.

5. The wireless intraocular pressure monitoring device as claimed in claim 2, wherein said reader unit includes:
a second wireless transceiver element; and
a frequency-to-voltage converter coupled electrically to said second wireless transceiver element for receiving the oscillation signal through said second wireless transceiver element, and operable to generate a converter output with a value corresponding to the frequency of the oscillation signal.

6. The wireless intraocular pressure monitoring device as claimed in claim 5, wherein said reader unit further includes
a power signal generator operable for generating a power signal, and coupled electrically to said second wireless transceiver element for wirelessly transmitting the power signal through said second wireless transceiver element.

7. The wireless intraocular pressure monitoring device as claimed in claim 6, wherein said wireless transceiver module further includes
a rectifier coupled electrically to said first wireless transceiver element for receiving the power signal through said first wireless transceiver element, and further coupled electrically to said oscillator for providing power to said oscillator.

8. The wireless intraocular pressure monitoring device as claimed in claim 5, wherein the converter output is an analog voltage output, and the value of the converter output is a magnitude of the analog voltage output,
said reader unit further including an output converter coupled electrically to said frequency-to-voltage converter, and configured to process the converter output so as to obtain the output signal.

9. The wireless intraocular pressure monitoring device as claimed in claim 8, wherein said output converter is configured to perform analog-to-digital conversion upon the converter output to result in a digital signal corresponding to the frequency of the oscillation signal, and to obtain the output signal based on the digital signal via one of a predefined look-up table and a predefined mathematical algorithm.

10. The wireless intraocular pressure monitoring device as claimed in claim 5, wherein said second wireless transceiver element is an antenna.

11. The wireless intraocular pressure monitoring device as claimed in claim 5, wherein said frequency-to-voltage converter includes:
a phase detector operable to receive the oscillation signal through said second wireless transceiver element, and to generate an error signal based on a difference in at least one of frequency and phase between the oscillation signal and a feedback signal;
a loop filter connected electrically to said phase detector to receive the error signal therefrom, and operable to filter out high frequency components and noise from the error signal so as to output a control voltage corresponding to the error signal, the control voltage serving as the converter output when the frequency and the phase of the oscillation signal match the frequency and the phase of the feedback signal, respectively; and
a voltage controlled oscillator connected electrically to said loop filter for receiving the control voltage from said loop filter, and operable to generate the feedback signal having a frequency and a phase that are dependent on the control voltage, and further coupled electrically to said phase detector for providing the feedback signal to said phase detector.

12. A sensor unit comprising:
a soft contact lens for wearing on a cornea such that a curvature of said soft contact lens corresponds substantially to that of the cornea;
an inductor embedded in said soft contact lens and having an inductance that corresponds to intraocular pressure when said soft contact lens is worn on the cornea; and
a wireless transceiver module coupled electrically to said inductor, and operable to generate an oscillation signal having a frequency dependent on the inductance of said inductor and to wirelessly transmit the oscillation signal.

13. The sensor unit as claimed in claim 12, wherein said wireless transceiver module includes:
a wireless transceiver element; and
an oscillator coupled electrically to said inductor, including a capacitor that is operatively associated with said inductor for generating the oscillation signal, and further coupled electrically to said wireless transceiver element for wirelessly transmitting the oscillation signal.

14. The sensor unit as claimed in claim 13, wherein said wireless transceiver module further includes
a rectifier coupled electrically to said wireless transceiver element for receiving a power signal through said wireless transceiver element, and further coupled electrically to said oscillator for providing power to said oscillator.

15. The sensor unit as claimed in claim 13, wherein said wireless transceiver element is an antenna.

16. A reader unit for wirelessly receiving an oscillation signal associated with intraocular pressure and for converting the oscillation signal into an output signal corresponding to the intraocular pressure, said reader unit comprising:
a wireless transceiver element; and
a frequency-to-voltage converter coupled electrically to said wireless transceiver element for receiving the oscillation signal through said wireless transceiver element, and operable to generate a converter output with a value corresponding to the frequency of the oscillation signal.

17. The reader unit as claimed in claim 16, further comprising
a power signal generator operable for generating a power signal, and coupled electrically to said wireless transceiver element for wirelessly transmitting the power signal through said second wireless transceiver element for receipt by a sensor unit that generates the oscillation signal.

18. The reader unit as claimed in claim 16, wherein the converter output is an analog voltage output, and the value of the converter output is a magnitude of the analog voltage output, said reader unit further comprising
an output converter coupled electrically to said frequency-to-voltage converter, and configured to process the converter output so as to obtain the output signal.

19. The reader unit as claimed in claim 18, wherein said output converter is configured to perform analog-to-digital conversion upon the converter output to result in a digital signal corresponding to the frequency of the oscillation signal, and to obtain the output signal based on the digital signal via one of a predefined look-up table and a predefined mathematical algorithm.

20. The reader unit as claimed in claim 16, wherein said wireless transceiver element is an antenna.

21. The reader unit as claimed in claim 16, wherein said frequency-to-voltage converter includes:
a phase detector operable to receive the oscillation signal through said wireless transceiver element, and to generate an error signal based on a difference in frequency and phase between the oscillation signal and a feedback signal;
a loop filter connected electrically to said phase detector to receive the error signal therefrom, and operable to filter out high frequency components and noise from the error signal so as to output a control voltage corresponding to the error signal, the control voltage serving as the converter output when the frequency and the phase of the oscillation signal match the frequency and the phase of the feedback signal, respectively; and
a voltage controlled oscillator connected electrically to said loop filter for receiving the control voltage from said loop filter, and operable to generate the feedback signal having a frequency and a phase that dependent on the control voltage, and further coupled electrically to said phase detector for providing the feedback signal to said phase detector.
